(19) United States
(12) Patent Application Publication
(10) Pub. No.: US 2026/0289908 A1
(43) Pub. Date: Sep. 24, 2026

(54) STATISTICAL STRUCTURE AND FUNCTION MODELING (SSFM) FOR PREDICTING MUSCULOSKELETAL INJURY RISKS

(71) Applicant: Southwest Research Institute, San Antonio, TX (US)

(72) Inventors: Daniel P. NICOLELLA, San Antonio, TX (US); Lance L. FRAZER, San Antonio, TX (US); Nathan LOUIS, San Antonio, TX (US); Gregory D. MYER, Flowery Branch, GA (US)

(21) Appl. No.: 19/461,868

(22) Filed: Jan. 28, 2026

Related U.S. Application Data
(60) Provisional application No. 63/750,744, filed on Jan. 28, 2025.

(51) Int. Cl. G06T 17/20; G06T 7/00; G06T 7/11

(52) U.S. Cl. CPC G06T 17/20; G06T 7/0012; G06T 7/11; G06T 2207/20081; G06T 2207/20084

(57) ABSTRACT

In an approach to prediction of musculoskeletal injury risks based on medical image data and biomechanics data, a system includes anatomical features determination circuitry; anatomical features variable reduction determination circuitry; biomechanics features determination circuitry; biomechanics features variable reduction determination circuitry; structure-function feature vector determination circuitry; predictive modeling circuitry; and a computing device. The system is configured to receive medical image data of one or more anatomical structures; receive biomechanics data of the one or more anatomical structures; generate one or more shape parameters from the medical image data; generate one or more biomechanics features from the biomechanics data; generate a structure-function feature vector from the one or more shape parameters combined with the one or more biomechanics features; perform predictive modeling to reduce a number of variables; and determine which structure-function features are different between an injured anatomical structure and a non-injured anatomical structure.

### CROSS-REFERENCE TO RELATED APPLICATIONS

The present application claims the benefit of the filing date of U.S. Provisional Application Ser. No. 63/750,744, filed Jan. 28, 2025, the entire teachings of which application is hereby incorporated herein by reference.

### FIELD

The present disclosure relates to statistical structure and function modeling (SSFM) for predicting musculoskeletal injury risks, and more particularly, to prediction of musculoskeletal injury risks based on medical image data and biomechanics data.

### BACKGROUND

Traditional methods focus on isolated measurements, such as muscle volume or other univariate anatomical measures (e.g., length, width, etc.) or univariate kinematic variables such as peak joint angle excursions during a prescribed task, which do not capture the full complexity of musculoskeletal injury risk. The SSFM integrates complex anatomical shape and traits with complex kinematic movement pattern data to provide a more holistic and sensitive model for predicting injuries across various parts of the body, improving injury prevention efforts.

Previous injury prediction systems rely heavily on isolated metrics like cross-sectional area or muscle strength, often failing to incorporate the comprehensive anatomical and biomechanical interactions that influence injury risk. The SSFM integrates multi-modal data types to significantly improve predictive accuracy.

### BRIEF DESCRIPTION OF THE DRAWINGS

Reference should be made to the following detailed description which should be read in conjunction with the following figures, wherein like numerals represent like parts.

FIG. 1 is a functional block diagram illustrating a system for SSFM for predicting musculoskeletal injury risks consistent with the present disclosure.

FIG. 2A is an example initial muscle surface mesh and FIG. 2B is the muscle surface mesh after isoparameterization consistent with the present disclosure.

FIG. 3 is a scree plot for combined muscle shape feature vector Principal Component Analysis (PCA) consistent with the present disclosure.

FIG. 4A is an example left perspective view and location of a muscle (biceps femoris) consistent with the present disclosure.

FIG. 4B is an example of an average muscle shape and size, an average muscle shape and size from an injured group, and an average muscle shape and size from a non-injured group consistent with the present disclosure.

FIG. 4C is an example cross-sectional area of the muscle as a function of muscle length for the injured group and the non-injured group consistent with the present disclosure.

FIG. 5 is an example of the differences in running kinematics between injured and non-injured groups consistent with the present disclosure.

FIG. 6A is an example of Area Under the Receiver Operating Characteristic (ROC) Curve (AUC) curves for each logistic regression analysis consistent with the present disclosure.

FIG. 6B is an example Mean ROC curves for each input in the neural network consistent with the present disclosure.

FIG. 7 is a flowchart diagram depicting operations for one illustrative example embodiment of the medical imaging process for the system for SSFM for predicting musculoskeletal injury risks consistent with the present disclosure.

FIG. 8 is a flowchart diagram depicting operations for one illustrative example embodiment of the biomechanics process for the system for SSFM for predicting musculoskeletal injury risks consistent with the present disclosure.

FIG. 9 is a flowchart diagram depicting operations for one illustrative example embodiment of the process to combine SFVs for the system for SSFM for predicting musculoskeletal injury risks consistent with the present disclosure.

FIG. 10 is a flowchart diagram depicting operations for one illustrative example embodiment of the process for combining the shape and kinematics vectors for the system for SSFM for predicting musculoskeletal injury risks consistent with the present disclosure.

FIG. 11 is a flowchart diagram depicting operations for one illustrative example embodiment of the process to determine injury probability for the system for SSFM for predicting musculoskeletal injury risks consistent with the present disclosure.

### DETAILED DESCRIPTION

Musculoskeletal injuries are a significant challenge in both high-performance sports and military contexts, leading to substantial costs and lost time. Traditional risk prediction methods typically focus on limited or univariate biomechanical or anatomical measures, such as muscle volume or single-plane kinematic variables, which lack the sensitivity to predict injury risk accurately.

A more comprehensive approach is needed-one that integrates both anatomical morphology and other anatomical characteristics with biomechanics to better assess injury risks. The Statistical Structure and Function Modeling (SSFM) system addresses this need by combining detailed anatomical data with detailed biomechanical/physiological function, providing robust injury prediction/disease risk model for a range of musculoskeletal injuries, including soft tissue injuries.

This invention relates to a new method of predicting musculoskeletal injury/disease risk using a combined Statistical Structure and Function Modeling (SSFM) approach. By integrating Statistical Shape Modeling (SSM) to capture anatomical morphology and Statistical Biomechanical/Function Modeling (SBM) to analyze functional patterns, the SSFM identifies key traits that contribute to injury risk. This system offers a significant improvement over traditional injury prediction methods by considering both anatomical structure and biomechanical function.

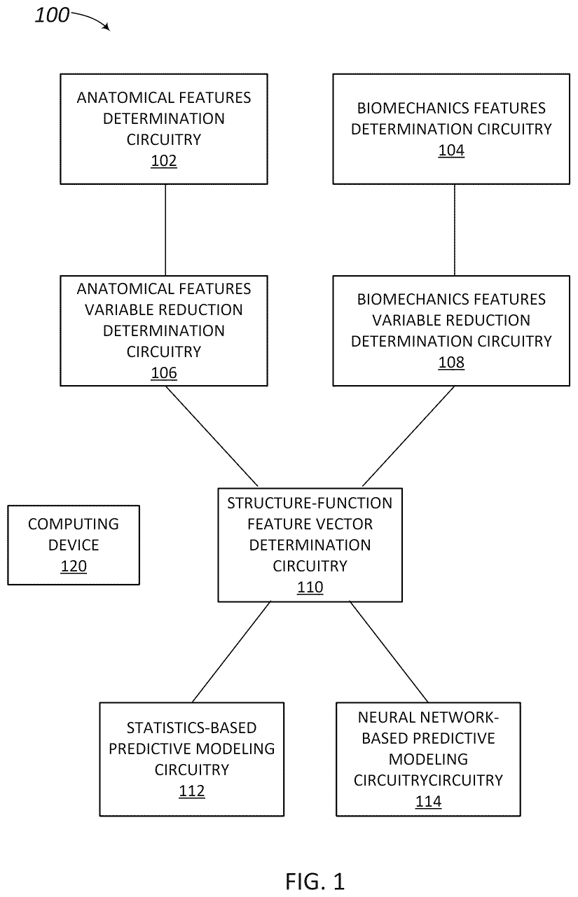

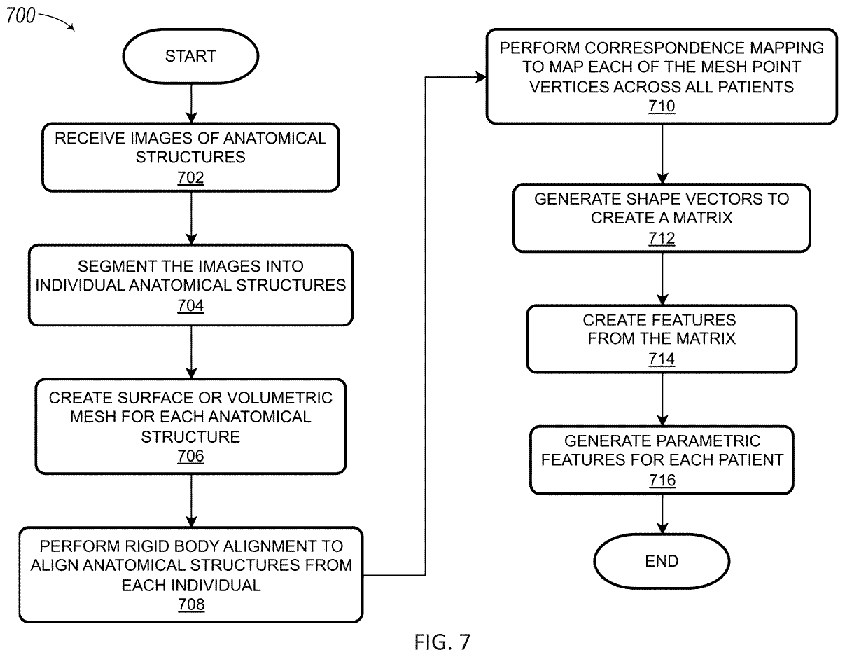

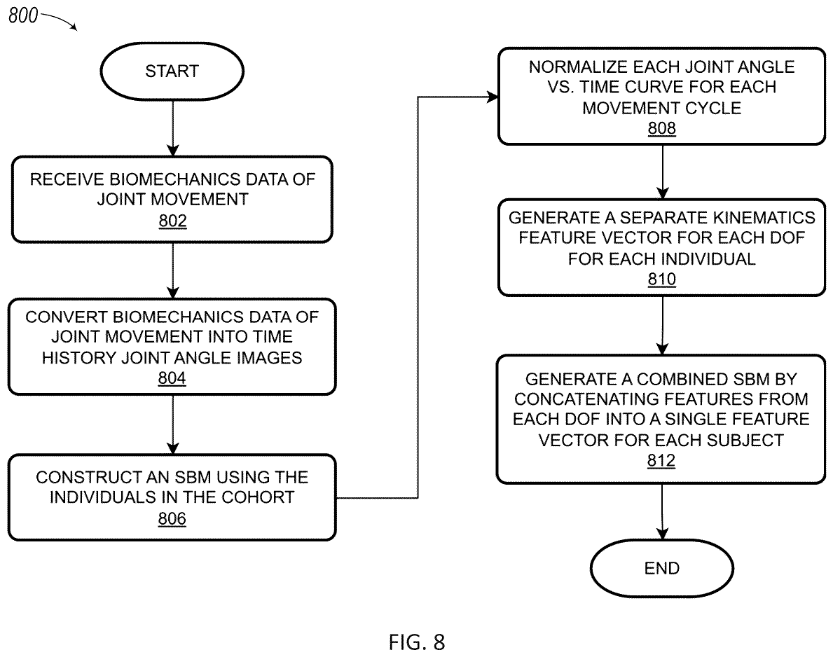

FIG. 1 is a functional block diagram illustrating a system 100 for SSFM for predicting musculoskeletal injury risks consistent with the present disclosure. The system 100 includes an anatomical features determination circuitry 102 and a biomechanics features determination circuitry 104. The output of the anatomical features determination circuitry 102 feeds into an anatomical features variable reduction determination circuitry 106, and the biomechanics features determination circuitry 104 feeds into a biomechanics features variable reduction determination circuitry 108. The operations for the anatomical features determination circuitry 102 and the anatomical features variable reduction determination circuitry 106 are described in FIG. 7 below. The operations for the biomechanics features determination circuitry 104 and the biomechanics features variable reduction determination circuitry 108 are described in FIG. 8 below.

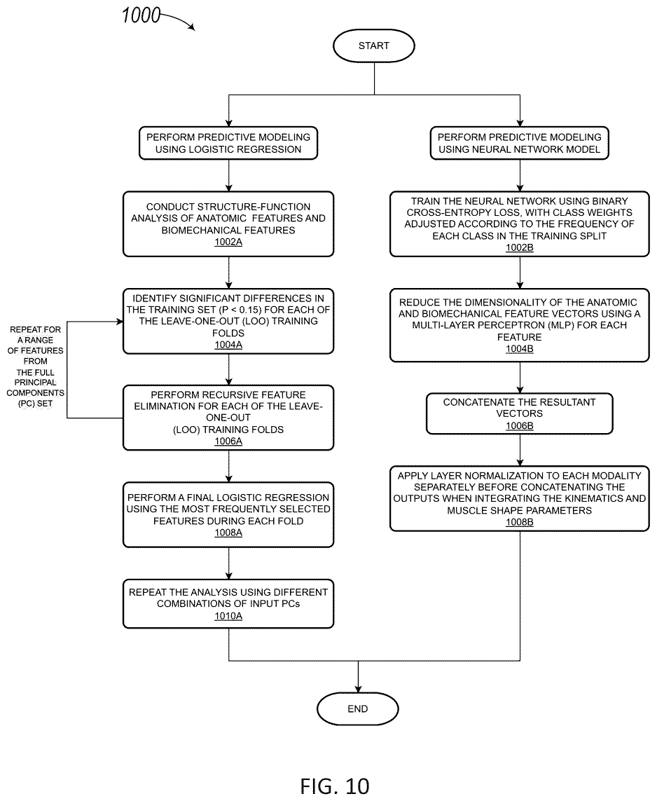

The anatomical features variable reduction determination circuitry 106 and the biomechanics features variable reduction determination circuitry 108 feed into a structure-function feature vector determination circuitry 110. The operations for the structure-function feature vector determination circuitry 110 are described in FIG. 10 below.

The output of the structure-function feature vector determination circuitry 110 is passed to one or more predictive models, such as statistics-based predictive modeling circuitry 112 and/or neural network-based predictive modeling circuitry 114. It should be noted, however, that other predictive modeling circuitry may be used as would be known to one skilled in the art. The operations for the statistics-based predictive modeling circuitry 112 and/or the neural network-based predictive modeling circuitry 114 are described in FIG. 10 below.

The system 100 also includes one or more computing devices 120. In an embodiment, computing device 120 can be a standalone computing device, a management server, a web server, a mobile computing device, or any other electronic device or computing system capable of receiving, sending, and processing data. In another embodiment, computing device 120 can represent a server computing system utilizing multiple computers as a server system, such as in a cloud computing environment. In yet another embodiment, computing device 120 represents a computing system utilizing clustered computers and components (e.g., database server computers, application server computers) that act as a single pool of seamless resources when accessed within system 100.

The SSFM model can be adapted for a variety of injury types across different parts of the musculoskeletal system, including injuries to the lower limbs, shoulders, back, and others. This method provides a tool for determining injury risk in individuals and improving injury prevention strategies in high-risk environments such as sports, military training, and industrial labor settings.

The SSFM integrates medical imaging and biomechanics/functional analyses to create a comprehensive injury risk profile. The model consists of three key components, anatomical morphology and property analysis (statistical shape and trait modeling), biomechanics/functional analysis (statistical biomechanical/functional modeling), and predictive modeling.

In the first component, anatomical The SSFM integrates medical imaging and biomechanics/functional analyses to create a comprehensive injury risk profile. The model consists of three key components, anatomical morphology and property analysis (statistical shape and trait modeling), biomechanics/functional analysis (statistical biomechanical/functional modeling), and predictive modeling morphology and property analysis (statistical shape and trait modeling), detailed parametric three-dimensional (3D) models of anatomical structures are generated from medical image data. This creates a parametric, corresponding feature vector that describes the shape of each anatomical structure in 3D space. In addition to shape, the spatial and volumetric distribution of tissue properties are parametrically mapped to the 3D shape model creating a shape and trait model. Variable reduction methods such as PCA, partial least squares, and/or other parametric representations as are known in the art, are used to identify variations in anatomical shape and traits between injured and non-injured individuals. These models can be surface based that only describe shape and they can be volumetric 3D models that include the shape combined with the 3D spatial distribution of tissue material characteristics (e.g., density, fat content, density, fiber angle, etc. derived from the medical image data). The disclosed methodology applies to both surface-based 3D models as well as volumetric 3D models. A flowchart diagram depicting operations for one illustrative example embodiment of the medical imaging process is shown in FIG. 7.

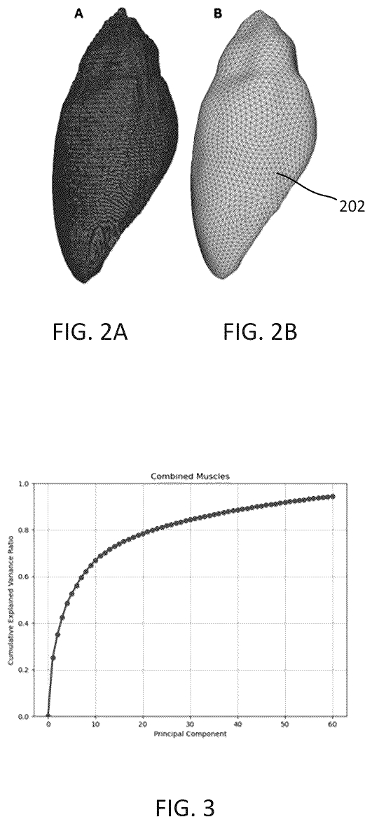

An example methodology is disclosed using surface-based parametric 3D anatomical structure models derived from magnetic resonance imaging (MRI). It should also be noted that the illustrative example used herein refers to a muscle, but the system and method may use any anatomical structure of interest. It should also be noted that the example methodology uses PCA for the parametric representation, but as previously mentioned, any other appropriate parametric representation could be used as would be know to one skilled in the art. Using established methods a statistical shape model (SSM) is created of the muscles from the segmented thigh MRI data of 84 subjects. 3D surface meshes were constructed from the muscle MRI segmentation data (see FIG. 2A). The segmentations and MRI image data are imported into an image processing software platform, e.g., Dragonfly from Object Research Systems, and the segmentations are converted to surface meshes and exported to software for processing and editing 3D triangular meshes, e.g., the open source Meshlab, for mesh processing (see FIG. 2B). In FIG. 2B, the surface meshes include a plurality of vertices 202. For each mesh, a uniform remeshing based on isoparameterization is applied with a sampling rate of four followed by Taubin smoothing.

A template mesh is selected (note, the same subject is used as the template for each muscle), and deformably registered to the remaining 83 subjects. Using an implementation of the Coherent Point Drift (CPD) algorithm for point cloud registration, such as the Python pycpd package, rigid registration with scaling is used to best align the template mesh and the target. Next, deformable registration is used to map the template nodes on the target mesh. Finally, the morphed template mesh is registered back to the original template position using singular value decomposition, such that no scaling occurs in the final registration step.

Next, muscle shape feature vectors were created as shown in Formula 1.

$${P}_{a} = {( {v}_{1 x} , {v}_{1 y} , {v}_{1 z} , … {v}_{i x} , {v}_{i y} , {v}_{i z} )}^{T} \qquad ( 1 )$$

In Formula 1, v_{i(xyz)} are the three-dimensional coordinates of the nodes in the muscle surface mesh, and α=1, . . . , 84, the total number of individuals in the example cohort, and T is transpose the row vector into a column vector. With 84 anatomically correspondent meshes for each muscle represented as muscle shape feature vectors, PCA is then used to parameterize the shape of each muscle. In addition, for each subject, each of the 14 muscles are combined into a single feature vector and variable reduction (e.g., PCA) is performed on the combined muscle shape feature vector. Independent, non-correlated principal components (PCs) derived from the variable reduction methods (e.g., PCA), or shape models are used to describe shape variability for each muscle and for the combined shape vector.

PCA is then applied to the muscle shape data to extract independent shape parameters describing variation across the study group. When combined into a single muscle shape feature vector, the first twenty PCs can explain approximately 80% of the variance within the group (see FIG. 3). FIG. 3 is a scree plot for combined muscle shape feature vector Principal Component Analysis (PCA). In FIG. 3, the horizontal (x-) axis is the principal component and the vertical (y-) axis is the cumulative explained variance ratio. Across individual muscles, fifteen significant PCs were identified as statistically different between the injured and non-injured groups. When muscle shape parameters were combined into a single shape feature vector, two PCs were found to be significantly different between the groups (Table 1). These findings suggest that specific variations in muscle morphology are associated with hamstring injury risk.

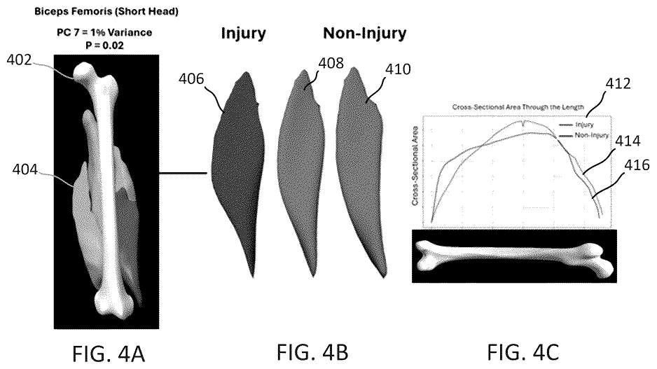

FIG. 4A is an example left perspective view and location of a muscle 404 (biceps femoris) and the associated femur bone 402 consistent with the present disclosure. FIG. 4B is an example of an average muscle shape and size 408, an average muscle shape and size from an injured group 406, and an average muscle shape and size from a non-injured group 410 consistent with the present disclosure. On average, the biceps femoris from the injured group is shorter and bulkier with less overall curvature while the average muscle from the non-injured group is longer and thinner with more curvature. FIG. 4C is a graph 412 of an example cross-sectional area of the muscle as a function of muscle length for the injured group and the non-injured group consistent with the present disclosure. In the graph 412 of FIG. 4C, the horizontal (x-) axis represents the cross-sectional area through the length of the muscle and the vertical (y-) axis represents the cross-sectional area of the muscle. The graph illustrates that, as shown in FIG. 4B, the injured muscle 414 tends to be shorter and bulkier with less overall curvature than the non-injured muscle.

The second key component of the model is biomechanics/functional analysis (statistical biomechanical/functional modeling). Functional data (e.g., kinematics, kinetics, EMF, EEG, etc.) collected during physical activities such as running, sprinting or any other activity are used to analyze functional output patterns. The functional data are time normalized using various key events (e.g., heal strike during running gait). Function feature vectors are constructed for each activity. These patterns are then linked to the risk of musculoskeletal injuries. A flowchart diagram depicting operations for one illustrative example embodiment of the biomechanics process is shown in FIG. 8.

An example application is given herein using kinematic data captured for each subject during running or sprinting as an example activity. The general approach can be applied to any activity analysis and any type of functional data capture system used to capture the subject activity (e.g., marker-based motion capture, wearable IMUs, markerless motion capture, EMG, EEG, etc.) and is not limited to those used in this example.

Each subject engaged in three trials of near-maximal effort sprinting, with each trial being progressively faster than the one before (initial, mid, and fast running speeds). These sprints were performed on an instrumented treadmill with kinematics recorded by video cameras. Once collected, data was batch processed using a markerless biomechanics software. The output of the markerless biomechanics software are the time history joint angles for the hip, knee, ankle, pelvis, torso, neck, shoulder, and elbow joints throughout the movement.

Following previously established methods, the 33 degree of freedom (dof) full body kinematic data were used to construct an SBM using the individuals in the cohort (n=84). Each joint angle versus time curve for each running gait cycle was normalized to 100 evenly spaced time points using consecutive right heel strike gait events to determine kinematic feature correspondence. The number of normalized time points can vary depending upon the movement captured. Subjects with kinematic data for at least two running gait cycles were included in this analysis (n=74; 9 injured, 65 uninjured). These data were used to generate a separate kinematics feature vector for each dof for each individual analogous to the muscle SSM disclosed above. In addition, a combined SBM was created by concatenating all kinematic dof's into a single feature vector for each subject. Similar to the muscle SSM, the running combined SBM feature vector for each individual was constructed as shown in Formula (2).

$${P}_{b} = {( {θ}_{11} , {θ}_{12} , {θ}_{1 n} , … {θ}_{i 1} , {θ}_{i 2} , {θ}_{i n} )}^{T} \qquad ( 2 )$$

In Formula 2, b=1 . . . 74 are the subjects with at least two gait cycles at each running speed, θ is the dof from ENABLE, i=1 . . . 33 dof representing a full body kinematic analysis, and n=1 . . . 100 normalized time points. To construct the SBM, PCA was performed on the separate kinematics feature vectors for each dof and for the combined kinematics feature vector to determine the set of uncorrelated parameters that describe the running kinematics of each subject.

Statistical analysis of the kinematic data identified several significant PCs that distinguished the injured group from the non-injured group. The first three significant PCs were derived from analyzing each dof separately, while the last significant PC was obtained from analyzing the entire kinematic vector (all dofs combined). The identified PCs suggest that specific kinematic patterns are associated with an increased risk of hamstring injury. These findings highlight the importance of kinematic and biomechanics profiles in injury risk and may guide future injury prevention strategies.

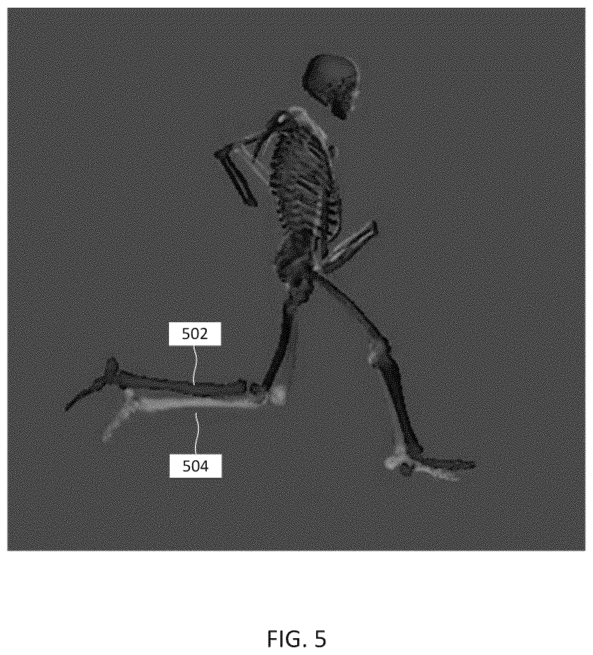

FIG. 5 is an example of the differences in running kinematics between injured 502 and non-injured 504 groups consistent with the present disclosure.

The third key component of the model is predictive modeling. In this component, machine learning algorithms (e.g., logistic regression, neural networks, etc.) are employed to integrate muscle shape data and biomechanical movement patterns, generating an injury risk prediction with high accuracy. Although the predictive modeling can be performed using only the muscle shape descriptors or a combination of muscle shape and kinematic descriptors, the disclosed system uses the combination of muscle shape and kinematic descriptors. A flowchart diagram depicting operations for one illustrative example embodiment of the predictive modeling process is shown in FIG. 10. Two types of example predictive modeling are disclosed herein.

The first type of predictive modeling is logistic regression. In logistic regression, a structure-function analysis, consisting of muscle anatomic structure combined with running kinematics, is conducted using all muscle shape PCs from the muscle statistical shape modeling and all gait PCs from the running kinematics analysis as inputs for a leave-one-out (LOO) logistic regression to predict future hamstring injuries. One illustrative example analysis included 74 subjects who had both gait and muscle data. For each of the 74 LOO training folds, feature selection was performed by first identifying significant differences in the training set (p<0.15), followed by recursive feature elimination. To determine the optimal number of features, the entire process was repeated within a for-loop, testing a range of 5 to 30 features from the full PC set. Since each training fold could select a different feature set, a final logistic regression was performed using the most frequently selected features during each fold. Additionally, the analysis was repeated using different combinations of input PCs: (1) separate muscle PCs combined with separate kinematic dof PCs, (2) separate muscle PCs combined with combined kinematic vector PCs, (3) combined muscle vector PCs with separate kinematic dof PCs, and (4) combined muscle vector PCs with combined kinematic vector PCs. The model's accuracy was assessed by reporting the average Area Under the Receiver Operating Characteristic (ROC) Curve (AUC).

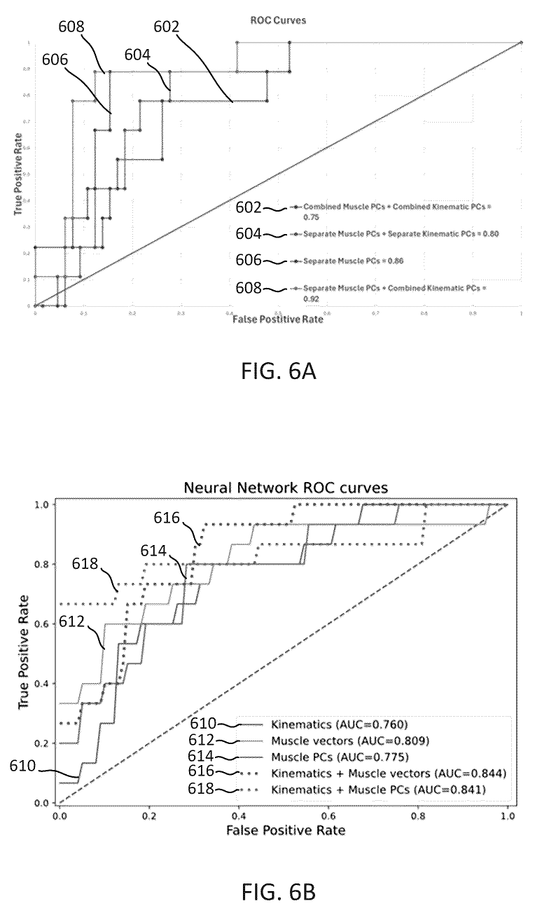

The logistic regression predictive models resulting in AUC values ranging from 0.75-0.92 depending on the features used in the model, as shown in the graph of FIG. 6A. In FIG. 6A, trace-1 602 shows that the combined muscle PCs plus the combined kinematic PCs equals 0.75, trace-2 604 shows that the separate muscle PCs plus the separate kinematic PCs equals 0.80, trace-3 606 shows that the separate muscle PCs equals 0.86, and trace-43 608 shows that the separate muscle PCs plus the combined kinematic PCs equals 0.92.

The most effective logistic regression model in predicting HSI was the combined structure-function model, incorporating separate muscle shape PCs and combined running kinematic PCs with an AUC of 0.92 (see graph 412 in FIG. 4C). This model, which represents muscle anatomical structure and running kinematics together, outperformed other configurations in predicting hamstring injuries.

The second type of predictive modeling is neural network modeling. In one illustrative example, this analysis evaluated the impact of combining running kinematics feature vectors at three different speeds, full muscle feature vectors, and muscle PCs on injury prediction. A five-fold cross-validation was used, with each fold consisting of 51 training subjects (6 injured) and 23 validation subjects (3 injured). The model's accuracy was assessed by reporting the average AUC across all validation folds.

The prediction model was based on a multi-layer perceptron (MLP), comprising three linear layers interspersed with two rectified linear unit (ReLU) activation functions. Prior to this, the dimensionality of the muscle vectors was reduced using a separate MLP (consisting of two linear layers and one ReLU) for each muscle, and the resultant vectors were concatenated. When integrating the kinematics and muscle shape parameters as inputs, layer normalization was applied to each modality separately before concatenating the outputs.

During model training, a grid search was conducted to identify the optimal learning rate and hidden dimension for each model. The problem was framed as a binary classification task, where training was performed using binary cross-entropy loss, with class weights adjusted according to the frequency of each class in the training split. For validation, the output of the prediction model was passed through a sigmoid activation function, and the highest average AUC across the five validation folds was reported.

The neural network analysis further supports the hypothesis that a structure-function model can effectively predict HSI (see the graph in FIG. 6B). In FIG. 6B, trace-5 610 shows that the kinematic AUC equals 0.760, trace-6 612 shows that the muscle vectors AUC equals 0.809, trace-7 614 shows that the muscle PCs AUC equals 0.775, trace-8 616 shows that the kinematic plus the muscle vectors AUC equals 0.844, and trace-8 618 shows that the kinematic plus the muscle PCs AUC equals 0.841.

For single input modalities, the muscle shape feature vectors alone, which represent the muscle anatomical structural component, provided the highest predictive power for hamstring injury, achieving an AUC of 0.809 while kinematics alone produced an AUC of 0.760 at the initial running speed. However, when combining structure (muscle vectors or muscle PCs) with function (running kinematics data), an increase in predictive performance is observed. The highest AUC (0.844) was achieved when combining fast-speed running kinematics feature vector with muscle shape feature vectors, indicating that integrating both structural and functional data enhances injury prediction accuracy. Additionally, the combination of middle-speed kinematics with muscle shape PCs yielded a comparable AUC of 0.841. These findings emphasize the importance of capturing both muscle morphology and running biomechanics in predicting hamstring injuries, reinforcing the value of a combined structure-function approach.

FIG. 7 is a flowchart diagram depicting operations for one illustrative example embodiment of the medical imaging process 700 for the system for SSFM for predicting musculoskeletal injury risks consistent with the present disclosure. It should be appreciated that embodiments of the present disclosure provide at least for the medical imaging process 700 for the system for SSFM for predicting musculoskeletal injury risks. However, FIG. 7 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 700 includes receiving images of anatomical structures (operation 702). In the illustrated example embodiment, the process 700 receives images of anatomical structures which may include, but are not limited to, MRI images, Computed Tomography (CT) images, ultrasound images, or any appropriate type of image as would be known to one skilled in the art.

Process 700 includes segmenting the images into individual anatomical structures (operation 704). In operation 704, the process 700 generates parametric 3D models of the anatomical structures from medical image data. This creates a parametric, corresponding feature vector that describes the shape of each anatomical structure in 3D space. In addition to shape, the spatial and volumetric distribution of tissue properties are parametrically mapped to the 3D shape model creating a shape and trait model.

Process 700 includes creating surface or volumetric mesh for each anatomical structure (operation 706). In operation 706, the process 700 3D constructs surface meshes from the segmentation data for the anatomical structures.

Process 700 includes performing rigid body alignment to align anatomical structures from each individual (operation 708). In operation 708, the process 700 aligns anatomical structures from each individual to remove spatial alignment variability, e.g., images of the same anatomical structures taken at different angles.

Process 700 includes performing correspondence mapping to map each of the mesh point vertices across all individuals (operation 710). In operation 710, the process 700 morphs α individual images, where α is the number of individuals, from each individual with images from all other individuals. This provides a correspondence maps, where each mesh point (i.e., each of the vertices from the mesh) represents the same relative anatomical position for each individual.

Process 700 includes generating shape vectors to create a matrix (operation 712). In operation 712, the process 700 generates shape vectors P_{α}. See Formula 1. This creates a matrix of the shape vectors for each of the a individuals.

Process 700 includes creating features from the matrix (operation 714). In the illustrated example embodiment of FIG. 7, in operation 714, the process 700 computes the eigenvalues and the eigenvectors of the matrix. The eigenvectors are individual shape variables that describe the shape variability of the anatomical structures for all of the individuals. The eigenvalues are the weight values for each individual, where the greater the eigenvalue means there are more variables described by the eigenvector. In other embodiments, other methods such as partial least squares or neural networks can also be used, as would be known by one skilled in the art.

Process 700 includes generating parametric features for each individual (operation 716).

Process 700 then ends for this cycle.

FIG. 8 is a flowchart diagram depicting operations for one illustrative example embodiment of the biomechanics process 800 for the system for SSFM for predicting musculoskeletal injury risks consistent with the present disclosure. It should be appreciated that embodiments of the present disclosure provide at least for the medical imaging process 800 for the system for SSFM for predicting musculoskeletal injury risks. However, FIG. 8 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 800 includes receiving biomechanics data of joint movement (operation 802). In the illustrated example embodiment, the process 800 receives videos of joint angles during movement for one or more individuals. In an embodiment, the biomechanics data may be kinematic videos. In some other embodiments, the biomechanics data may come from any measurement modality such as maker-based, markerless, wearable, etc.

Process 800 includes converting the biomechanics data of joint movement into time history joint angle images (operation 804). In operation 804, the process 800 processes the video data using markerless biomechanics software. The output of the markerless biomechanics software system is, for this illustrative example, the time history joint angles for the hip, knee, ankle, pelvis, torso, neck, shoulder, and elbow joints throughout the movement.

Process 800 includes constructing an SBM using the individuals in the cohort (operation 806). In operation 806, the process 800 uses the degree of freedom (dof) full body kinematic data to construct a statistical biomechanics model (SBM) using the individuals in the cohort (in the illustrative example presented here, n=84 individuals).

Process 800 includes normalizing each joint angle vs. time curve for each movement cycle (operation 808). In operation 808, the process 800 normalizes each joint angle versus time curve for each movement cycle to 100 evenly spaced time points using, for example, consecutive right heel strike gait events to determine kinematic feature correspondence. The number of normalized time points can vary depending upon the movement captured.

Process 800 includes generating a separate kinematics feature vector for each dof for each individual (operation 810). In operation 810, the process 800 generates a separate kinematics feature vector for each dof for each individual analogous to the muscle SSM described above. See Formula 2.

Process 800 includes generating a combined SBM by concatenating features from each dof into a single feature vector for each individual (operation 812). In operation 812, the process 800 a combined SBM was created by concatenating all kinematic dof's into a single feature vector for each subject.

Process 800 then ends for this cycle.

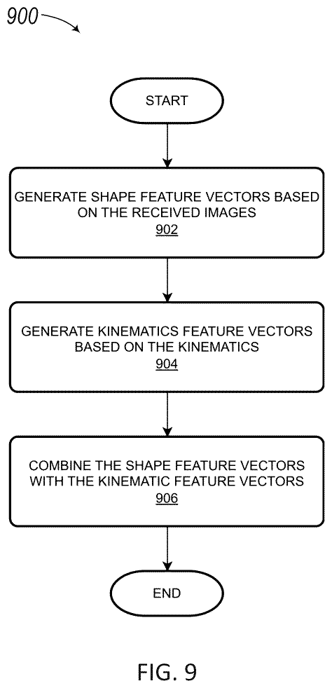

FIG. 9 is a flowchart diagram depicting operations for one illustrative example embodiment of the process 900 to combine SFVs for the system for SSFM for predicting musculoskeletal injury risks consistent with the present disclosure. It should be appreciated that embodiments of the present disclosure provide at least for the process 900 to combine SFVs for the system for SSFM for predicting musculoskeletal injury risks. However, FIG. 9 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 900 includes generating shape feature vectors based on the received images (operation 902).

Process 900 includes generating kinematics feature vectors based on the kinematics (operation 904).

Process 900 includes combining the shape feature vectors with the kinematic feature vectors (operation 906).

Process 900 then ends for this cycle.

FIG. 10 is a flowchart diagram depicting operations for one illustrative example embodiment of the process 1000 for combining the shape and kinematics vectors for the system for SSFM for predicting musculoskeletal injury risks consistent with the present disclosure. It should be appreciated that embodiments of the present disclosure provide at least for the process 1000 for combining the shape and kinematics vectors for the system for SSFM for predicting musculoskeletal injury risks. However, FIG. 10 provides only an illustration of one implementation and does not imply any limitations with regard to the environments in which different embodiments may be implemented. Many modifications to the depicted environment may be made by those skilled in the art without departing from the scope of the disclosure as recited by the claims.

In one illustrative embodiment, the process 1000 may perform predictive modeling using logistic regression (see operations 1002A-1010A). In another illustrative embodiment, the process 1000 may perform predictive modeling using neural network modeling (see operations 1002B-1008B). In other embodiments, the process 1000 may use any appropriate method of predictive modeling as would be known to one skilled in the art. For clarity, the logistic regression and neural network modeling embodiments are described below.

Process 1000 includes conducting structure-function analysis of anatomic features and biomechanical features (operation 1002A). In the illustrated example embodiment, a structure-function analysis, consisting of anatomic features combined with biomechanical features, is conducted using all anatomic features shape PCs from the anatomic features statistical shape modeling and all gait PCs from the running biomechanical analysis as inputs for a leave-one-out (LOO) logistic regression to predict future injuries, e.g., hamstring injuries.

Process 1000 includes identifying significant differences in the training set for each of the leave-one-out (LOO) training folds (operation 1004A). In operation 1004A, for each of the LOO 74 training folds, the process 1000 performs feature selection each of the LOO training folds by first identifying significant differences in the training set, e.g., p<0.15, followed by recursive feature elimination.

Process 1000 includes performing recursive feature elimination for each of the LOO training folds (operation 1006A).

Operations 1004A and 1006A repeat for a range of features from the full principal components (PC) set. To determine the optimal number of features, the entire process is repeated within a for-loop, testing a range of, for example, 5 to 30 features from the full PC set.

Process 1000 includes performing a final logistic regression using the most frequently selected features during each fold (operation 1008A).

Process 1000 includes repeating the analysis using different combinations of input PCs (operation 1010A). In operation 1010A, the process 1000 repeats the analysis using different combinations of input PCs which may include, but are not limited to, (1) separate muscle PCs combined with separate kinematic dof PCs, (2) separate muscle PCs combined with combined kinematic vector PCs, (3) combined muscle vector PCs with separate kinematic dof PCs, and (4) combined muscle vector PCs with combined kinematic vector PCs. The model's accuracy is assessed by reporting the average Area Under the Receiver Operating Characteristic (ROC) Curve (AUC).

For predictive modeling using logistic regression, process 1000 ends for this cycle.

Process 1000 includes training the neural network using binary cross-entropy loss, with class weights adjusted according to the frequency of each class in the training split (operation 1002B). In operation 1002B, the process 1000 validates the output of the prediction model by passing it through a sigmoid activation function, and the highest average AUC across the five validation folds is reported.

Process 1000 includes reducing the dimensionality of the anatomic and biomechanical feature vectors using a multi-layer perceptron (MLP) for each feature (operation 1004B). In operation 1004B, the process 1000 uses a prediction model based on a multi-layer perceptron (MLP), comprising three linear layers interspersed with two rectified linear unit (ReLU) activation functions. Prior to this, the dimensionality of the muscle vectors is reduced using a separate MLP (consisting of two linear layers and one ReLU) for each muscle, and the resultant vectors are concatenated. When integrating the kinematics and muscle shape parameters as inputs, layer normalization is applied to each modality separately before concatenating the outputs.

Process 1000 includes concatenating the resultant vectors (operation 1006B).

Process 1000 includes applying layer normalization to each modality separately before concatenating the outputs when integrating the kinematics and muscle shape parameters (operation 1008B).

For predictive modeling using neural network modeling, process 1000 ends for this cycle.

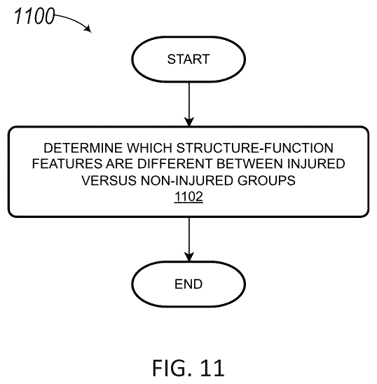

FIG. 11 is a flowchart diagram depicting operations for one illustrative example embodiment of the process to determine injury probability for the system for SSFM for predicting musculoskeletal injury risks consistent with the present disclosure. It should be appreciated that embodiments of the present disclosure provide at least for the process to determine injury probability for the system for SSFM for predicting musculoskeletal injury risks. However, FIG. 11 provides only an illustration of one implementation and does not imply any limitations with regard to the environments in which different embodiments may be implemented. Many modifications to the depicted environment may be made by those skilled in the art without departing from the scope of the disclosure as recited by the claims.

Process 1100 includes determining which structure-function features are different between injured versus non-injured anatomical structures (operation 1102). In an embodiment, process 1100 may determine which structure-function features are different between injured versus non-injured anatomical structures using statistical testing, ML, ML/Artificial intelligence (AI) based clustering, etc., as would be known to one skilled in the art.

Process 1100 then ends for this cycle.

According to one aspect of the disclosure there is thus provided a system for prediction of musculoskeletal injury risks based on medical image data and biomechanics data. The system includes anatomical features determination circuitry; anatomical features variable reduction determination circuitry; biomechanics features determination circuitry; biomechanics features variable reduction determination circuitry; structure-function feature vector determination circuitry; predictive modeling circuitry; and a computing device. The system is configured to receive medical image data of one or more anatomical structures; receive biomechanics data of the one or more anatomical structures; generate one or more shape parameters from the medical image data; generate one or more biomechanics features from the biomechanics data; generate a structure-function feature vector from the one or more shape parameters combined with the one or more biomechanics features; perform predictive modeling to reduce a number of variables; and determine which structure-function features are different between an injured anatomical structure and a non-injured anatomical structure.

According to another aspect of the disclosure, there is thus provided a method of prediction of musculoskeletal injury risks based on medical image data and biomechanics data. The method includes receiving medical image data of one or more anatomical structures; receiving biomechanics data of the one or more anatomical structures; generating one or more shape parameters from the medical image data; generating one or more biomechanics features from the biomechanics data; generating a structure-function feature vector from the one or more shape parameters combined with the one or more biomechanics features; performing predictive modeling to reduce a number of variables; and determining which structure-function features are different between an injured anatomical structure and a non-injured anatomical structure.

The embodiments and examples described herein may be implemented using, for example, software (e.g., instruction sets) and various hardware components (e.g., circuitry, sensors, processors, etc.), data and data sets (for example, medical imaging data, etc.) to achieve the advantages and features described herein.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

Artificial intelligence (AI) as used herein is generally defined as the theory and development of computer systems able to perform tasks that normally require human intelligence, such as speech recognition, visual perception, decision-making, and translation between languages. The term AI is often used to describe systems that mimic cognitive functions of the human mind, such as learning and problem solving.

Machine learning (ML) is an application of AI that creates systems that have the ability to automatically learn and improve from experience. ML involves the development of computer programs that can access data and learn based on that data. ML algorithms typically build mathematical models based on sample, or training, data in order to make predictions or decisions without being explicitly programmed to do so. The use of training data in ML requires human intervention for feature extraction in creating the training data set. The two main types of ML are Supervised learning and Unsupervised learning. Supervised learning uses labeled datasets that are designed to train or “supervise” algorithms into classifying data or predicting outcomes accurately. Supervised learning is typically used for problems requiring classification or regression analysis. Classification problems use an algorithm to accurately assign test data into specific categories. Regression is a method that uses an algorithm to understand the relationship between dependent and independent variables. Regression models are helpful for predicting numerical values based on different data points.

Unsupervised learning uses machine learning algorithms to analyze and cluster unlabeled datasets. These algorithms discover hidden patterns or data groupings without the need for human intervention, and their ability to discover similarities and differences in information make unsupervised learning the ideal solution for exploratory data analysis, cross-selling strategies, customer segmentation, and image recognition. Unsupervised learning is typically used for problems requiring clustering, e.g., K-means clustering, or association, which uses different rules to find relationships between variables in a given dataset.

Deep learning is a sub-field of ML that automates much of the feature extraction, eliminating some of the manual human intervention required and enabling the use of larger data sets. Deep learning typically uses neural networks, which are highly interconnected entities, called nodes. Each node, or artificial neuron, connects to another and has an associated weight and threshold. A node multiplies the input data with the weight, which either amplifies or dampens that input, thereby assigning significance to inputs with regard to the task the algorithm is trying to learn. If the output of any individual node is above the specified threshold value, that node is activated, sending data to the next layer of the network. Otherwise, no data is passed along to the next layer of the network. A neural network that consists of more than three layers can be considered a deep learning algorithm or a deep neural network.

As used in any embodiment herein, the terms “system” and/or “circuit” may refer to, for example, software, firmware, and/or circuitry configured to perform any of the aforementioned operations. Software may be embodied as a software package, code, instructions, instruction sets and/or data recorded on non-transitory, computer-readable storage devices. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry and/or future computing circuitry including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

Any of the operations described herein may be implemented in a system that includes one or more non-transitory storage devices having stored therein, individually or in combination, instructions that when executed by circuitry perform the operations. Here, the circuitry may include any of the aforementioned circuitry including, for example, one or more processors, ASICS, ICs, etc., and/or other programmable circuitry. Also, it is intended that operations described herein may be distributed across a plurality of physical devices, such as processing structures at more than one different physical location. The storage device includes any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), embedded multimedia cards (eMMCs), secure digital input/output (SDIO) cards, magnetic or optical cards, or any type of media suitable for storing electronic instructions. Other embodiments may be implemented as software executed by a programmable control device.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents. Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

What is claimed is:

1. A system for prediction of musculoskeletal injury risks based on medical image data and biomechanics data, the system comprising:
anatomical features determination circuitry;
anatomical features variable reduction determination circuitry;
biomechanics features determination circuitry;
biomechanics features variable reduction determination circuitry;
structure-function feature vector determination circuitry;
predictive modeling circuitry; and
a computing device;
the system configured to:
receive medical image data of one or more anatomical structures;
receive biomechanics data of the one or more anatomical structures;
generate one or more shape parameters from the medical image data;
generate one or more biomechanics features from the biomechanics data;
generate a structure-function feature vector from the one or more shape parameters combined with the one or more biomechanics features;
perform predictive modeling to reduce a number of variables; and
determine which structure-function features are different between an injured anatomical structure and a non-injured anatomical structure.

2. The system of claim 1, wherein generate the one or more shape parameters from the medical image data further comprises:
segment the medical image data;
create a surface mesh or a volume mesh for each of the one or more anatomical structures;
perform rigid body alignment to align the one or more anatomical structures from each of a plurality of individuals;
perform correspondence mapping to map each of a plurality of mesh point vertices of the surface mesh for each of the one or more anatomical structures for each of the plurality of individuals;
generate a shape vector for each of the plurality of individuals to create a matrix;
compute eigenvalues and eigenvectors of the matrix; and
generate one or more parametric features for each of the plurality of individuals.

3. The system of claim 2, wherein segment the medical image data further comprises:
generate one or more parametric three-dimensional (3D) models of the one or more anatomical structures from the medical image data to create a parametric corresponding feature vector, wherein the parametric corresponding feature vector describes a shape of each one or more anatomical structures in a 3D space.

4. The system of claim 3, wherein create the surface mesh for each of the one or more anatomical structures further comprises:
convert the one or more parametric 3D models of the one or more anatomical structures to one or more surface meshes.

5. The system of claim 4, further comprising:
apply a uniform remeshing based on isoparameterization to the one or more surface meshes; and
apply a Taubin smoothing to the uniform remeshing.

6. The system of claim 5, wherein the isoparameterization uses a sampling rate of four.

7. The system of claim 2, wherein generate one or more biomechanics features from the biomechanics data further comprises:
convert biomechanics data of joint movement into one or more time history joint angle images;
construct an statistical biomechanics model (SBM) for the plurality of individuals;
normalize each of joint angle of the one or more time history joint angle images versus a time curve for one or more movement cycles;
generate a separate kinematics feature vector for each degree of freedom (dof) for each of the plurality of individuals; and
generate a combined SBM by concatenating a plurality of features from each dof into a single feature vector for each of the plurality of individuals.

8. The system of claim 1, wherein perform the predictive modeling uses logistic regression.

9. The system of claim 8, wherein the logistic regression comprises:
conduct structure-function analysis of one or more anatomical features and one or more biomechanics features;
identify one or more significant differences in a training set for each of one or more leave-one-out (LOO) training folds for a range of features from a full principal components (PC) set;
perform recursive feature elimination for each of the one or more LOO training folds for the range of features from the full PC set;
perform a final logistic regression using one or more most frequently selected features during each fold; and
repeat the structure-function analysis using different combinations of input PCs.

10. The system of claim 1, wherein perform the predictive modeling uses neural network modeling.

11. The system of claim 10, wherein the neural network modeling comprises:
train a neural network using binary cross-entropy loss, wherein one or more class weights are adjusted according to a frequency of each of a plurality of classes in a training split;
reduce a dimensionality of a anatomical feature vector and a biomechanics feature vector using a multi-layer perceptron (MLP) for each of a plurality of features from the anatomical feature vector and the biomechanics feature vector;
concatenate one or more resultant vectors; and
apply a layer normalization to each modality separately before concatenating one or more outputs of the MLP when integrating the biomechanics features and muscle shape parameters.

12. A method for prediction of musculoskeletal injury risks based on medical image data and biomechanics data, the method comprising:
receiving medical image data of one or more anatomical structures;
receiving biomechanics data of the one or more anatomical structures;
generating one or more shape parameters from the medical image data;
generating one or more biomechanics features from the biomechanics data;
generating a structure-function feature vector from the one or more shape parameters combined with the one or more biomechanics features;
performing predictive modeling to reduce a number of variables; and
determining which structure-function features are different between an injured anatomical structure and a non-injured anatomical structure.

13. The method of claim 12, wherein generating the one or more shape parameters from the medical image data further comprises:
segmenting the medical image data;
creating a surface mesh for each of the one or more anatomical structures;
performing rigid body alignment to align the one or more anatomical structures from each of a plurality of individuals;
performing correspondence mapping to map each of a plurality of mesh point vertices of the surface mesh for each of the one or more anatomical structures for each of the plurality of individuals;
generating a shape vector for each of the plurality of individuals to create a matrix;
computing eigenvalues and eigenvectors of the matrix; and
generating one or more parametric features for each of the plurality of individuals.

14. The method of claim 13, wherein segmenting the medical image data further comprises:
generating one or more parametric three-dimensional (3D) models of the one or more anatomical structures from the medical image data to create a parametric corresponding feature vector, wherein the parametric corresponding feature vector describes a shape of each one or more anatomical structures in a 3D space.

15. The method of claim 14, wherein creating the surface mesh for each of the one or more anatomical structures further comprises:
converting the one or more parametric 3D models of the one or more anatomical structures to one or more surface meshes.

16. The method of claim 15, further comprising:
applying a uniform remeshing based on isoparameterization to the one or more surface meshes; and
applying Taubin smoothing to the uniform remeshing.

17. The system of claim 16, wherein the isoparameterization uses a sampling rate of four.

18. The method of claim 13, wherein generating one or more biomechanics features from the biomechanics data further comprises:
converting biomechanics data of joint movement into one or more time history joint angle images;
constructing an statistical biomechanics model (SBM) for the plurality of individuals;
normalizing each of joint angle of the one or more time history joint angle images versus a time curve for one or more movement cycles;
generating a separate kinematics feature vector for each degree of freedom (dof) for each of the plurality of individuals; and
generating a combined SBM by concatenating a plurality of features from each dof into a single feature vector for each of the plurality of individuals.

19. The method of claim 12, wherein the predictive modeling comprises:
conducting structure-function analysis of one or more anatomical features and one or more biomechanics features;
identifying one or more significant differences in a training set for each of one or more leave-one-out (LOO) training folds for a range of features from a full principal components (PC) set;
performing recursive feature elimination for each of the one or more LOO training folds for the range of features from the full PC set;
performing a final logistic regression using one or more most frequently selected features during each fold; and
repeating the structure-function analysis using different combinations of input PCs.

20. The method of claim 12, wherein the predictive modeling comprises:
training a neural network using binary cross-entropy loss, wherein one or more class weights are adjusted according to a frequency of each of a plurality of classes in a training split;
reducing a dimensionality of a anatomical feature vector and a biomechanics feature vector using a multi-layer perceptron (MLP) for each of a plurality of features from the anatomical feature vector and the biomechanics feature vector;
concatenating one or more resultant vectors; and
applying a layer normalization to each modality separately before concatenating one or more outputs of the MLP when integrating the biomechanics features and muscle shape parameters.
